(19) United States
(12) Patent Application Publication
(10) Pub. No.: US 2022/0213421 A1
(43) Pub. Date: Jul. 7, 2022

(54) DEVICE FOR GROWING MICROORGANISMS

(71) Applicant: 3M INNOVATIVE PROPERTIES COMPANY, St. Paul, MN (US)

(72) Inventors: Alexi J. Young, Shoreview, MN (US); Kurt J. Halverson, Lake Elmo, MN (US); Steven P. Swanson, Blaine, MN (US); Evan D. Brutinel, Inver Grove Heights, MN (US); Caleb T. Nelson, McKinney, TX (US)

(21) Appl. No.: 17/610,670

(22) PCT Filed: May 29, 2020
(86) PCT No.: PCT/IB2020/055126

§ 371 Date: Nov. 11, 2021

Related U.S. Application Data
(60) Provisional application No. 62/866,380, filed on Jun. 25, 2019.

(51) Int. Cl. C12M 3/06; C12Q 1/10; C12N 1/20; C12M 1/00; C12M 1/12; C12M 1/34

(52) U.S. Cl. CPC C12M 23/16; C12Q 1/10; C12N 1/20; C12M 41/00; C12M 23/22; C12M 23/38; C12M 25/00; C12M 23/20

(57) ABSTRACT

A device for growing microorganisms. The device includes a body member comprising a self-supporting, water-proof substrate having upper and lower surfaces; a hydrophobic spacer element adhered to the upper surface of the substrate forming side walls to retain a predetermined amount of liquid in contact with the substrate, wherein the hydrophobic spacer element has a hole therein; a fluid control film in the hole of the hydrophobic spacer element; a cover sheet having an inner-facing surface and an outer-facing surface, the cover sheet adhered to at least a portion of the body member; and a substantially dry, first microbial growth nutrient composition disposed on a portion of the inner surface of the cover sheet; a first adhesive composition adhered to the first microbial growth nutrient composition; and a cold-water-soluble first hydrogel-forming composition adhered to the first adhesive composition.

### BACKGROUND

A wide variety of culture devices have been developed. As one example, culture devices have been developed by 3M Company (hereafter “3M”) of St. Paul, Minn. In particular, culture devices are sold by 3M under the trade name PETRIFILM plates. Culture devices can be utilized to facilitate the rapid growth and detection of microorganisms commonly associated with food contamination, including, for example, aerobic bacteria, E. coli., coliforms, enterobacteria, yeast, mold, Staphylococcus aureus, Listeria, Campylobacter, and the like. The use of PETRIFILM plates, or other growth media, can simplify bacterial testing of food samples, for instance.

Culture devices can be used to enumerate or identify the presence of bacteria so that corrective measures can be performed (in the case of food testing) or proper diagnosis can be made (in the case of medical use). In other applications, culture devices may be used to rapidly grow microorganisms in laboratory samples, e.g., for experimental purposes.

### SUMMARY

Devices and methods for the propagation or storage of microorganisms are provided.

Thus, in one aspect, the present disclosure provides a device for growing microorganisms. The device includes a body member comprising a self-supporting, water-proof substrate having upper and lower surfaces; a hydrophobic spacer element adhered to the upper surface of the substrate forming side walls to retain a predetermined amount of liquid in contact with the substrate, wherein the hydrophobic spacer element has a hole therein; a fluid control film in the hole of the hydrophobic spacer element; a cover sheet having an inner-facing surface and an outer-facing surface, the cover sheet adhered to at least a portion of the body member; and a substantially dry, first microbial growth nutrient composition disposed on a portion of the inner surface of the cover sheet; a first adhesive composition adhered to the first microbial growth nutrient composition; and a cold-water-soluble first hydrogel-forming composition adhered to the first adhesive composition.

In another aspect, the present disclosure provides a method. The method includes providing a device of the current disclosure; adding a predetermined volume of a sample containing at least one microorganism into the device to form an inoculated device; contacting the cover sheet to the self-supporting, water-proof substrate; incubating the inoculated device; and detecting the presence or an absence of a colony of the target microorganism in the device.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### Definitions

For the following defined terms, these definitions shall be applied for the entire Specification, including the claims, unless a different definition is provided in the claims or elsewhere in the Specification based upon a specific reference to a modification of a term used in the following definitions:

The terms “about” or “approximately” with reference to a numerical value or a shape means +/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

The term “a”, “an”, and “the” are used interchangeably with “at least one” to mean one or more of the elements being described.

The term “and/or” means either or both. For example, the expression “A and/or B” means A, B, or a combination of A and B.

“Cluster” refers to a group of agglomerated and/or aggregated particles.

“Agglomerated” refers to a weak association of primary particles or aggregated particles usually held together by charge or polarity. Agglomerated particles can typically be broken down into smaller entities by, for example, shearing forces encountered during dispersion of the agglomerated particles in a liquid. The terms “aggregated” and “aggregates” refer to a strong association of primary particles often bound together by, for example, residual chemical treatment, covalent chemical bonds, or ionic chemical bonds. Further breakdown of the aggregates into smaller entities is very difficult to achieve.

“Cold-water-soluble” refers to material which forms a solution in water at room temperature (i.e., about 25° C.).

“Hydrophobic” refers to a material that exhibits a water contact angle of 90° or larger on its surface.

“Opaque” refers to a substrate having at most 10% light transmission.

“Powder” refers to a finely divided particulate material having an average diameter in a range from 0.1 micrometer up to 400 micrometers.

“Reconstituted medium” refers to a solution or gel formed from the reconstitution of a cold-water-soluble powder with an aqueous liquid.

“Substantially impermeable to microorganisms and water vapor”, as used herein, refers to a cover sheet that prevents undesired contamination and hydration of underlying layers of cold-water-soluble powder during shipping, storage, and use of thin film culture device(s), and avoids desiccation of the reconstituted medium, such that the reconstituted medium is suitable to support the growth of microorganisms during an incubation period.

“Substantially water-free”, as used herein, designates a water content no greater than about the water content of the ambient environment.

“Test sample”, as used herein, refers to a component or portion taken from a food product, a human or animal test subject, pharmaceutical or cosmetic commodity, soil, water, air or other environmental source, or any other source from which a presence and, optionally, an enumeration of aerobic and/or aerotolerant bacteria is to be determined. A test sample may be taken from a source using techniques known to one skilled in the art including, for example, pouring, pipetting, swabbing, filtering, and contacting. In addition, the test sample may be subjected to various sample preparation processes known in the art including, for example, blending, stomaching, homogenization, enrichment, selective enrichment, or dilution.

“Transparent” refers to a substrate having at least 90% light transmission.

### BRIEF DESCRIPTION OF THE DRAWINGS

The disclosure may be more completely understood in consideration of the following detailed description of various embodiments of the disclosure in connection with the accompanying figures, in which:

FIG. 1 is a top perspective view, partially in section, of yet another exemplary device according to the present disclosure.

FIG. 2 is a schematic illustration of a channeled microstructured surface of the present disclosure with a quantity of fluid thereon.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this disclosure.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

As used in this Specification, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

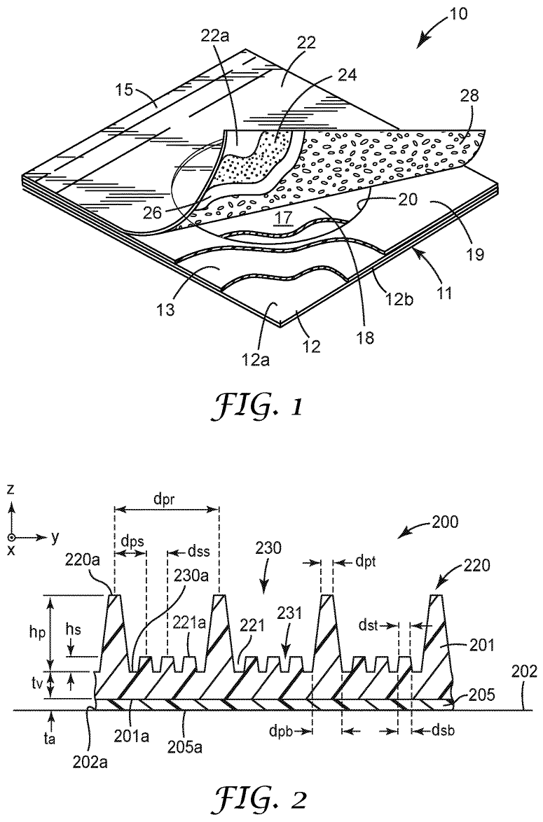

FIG. 1 illustrates an exemplary embodiment of a device for growing microorganisms. The device 10 includes a body member 11 including a substrate 12 having a first major surface 12a (e.g., upper surface) and a second major surface 12b (e.g., lower surface) and a cover sheet 22 attached to at least a portion of the body member 11, where the cover sheet 22 includes a first major surface 22a (e.g., inner surface) facing the body member 11. The device 10 further includes a substantially dry, first microbial growth nutrient composition 24 disposed on a portion of the first major surface 22a of the cover sheet 22, a first adhesive composition 26 adhered to the first microbial growth nutrient composition 24, and a cold-water-soluble first hydrogel-forming composition 28 adhered to the first adhesive composition 26. Preferably, the device also includes a hydrophobic spacer element 19 disposed on the first major surface 12a of the substrate 12. In general, the spacer element 19 comprises a water-insoluble substrate defining a hole or aperture 20. The spacer element 19 can be a hydrophobic foam sheet, for example, polystyrene or polyethylene foam sheet. In use, a user separates the cover sheet 22 from the substrate 12 sufficiently to add an amount of a sample containing at least one microorganism within hole or aperture 20 defined by the spacer 19, places the cover sheet 22 back in contact with the substrate 12 to form an inoculated device, and incubates the inoculated device. The area on the first major surface 12a of the substrate 12 defined by the aperture 20 may also be referred to as a sample-receiving zone 17. A fluid control film 18 can be disposed in the hole of the hydrophobic spacer element 19 and on the first major surface 12a of substrate 12. The device 10 may further a second adhesive composition 13 adhered to the upper surface 12a of the self-supporting waterproof substrate 12 and the second adhesive composition 13 is in between the hydrophobic spacer element 19 and the substrate 12.

The aperture 20 can be any shape. Non-limiting examples of useful shapes for the aperture 20 include a square, a rectangle, a circle, an oval, a polygon, a hexagon, and an octagon. The area of the sample-receiving zone (and aperture 20) may be selected based on, for example, the volume of sample (e.g., aqueous liquid) to be deposited in the zone. In any embodiment, for a 0.5-3 milliliter sample, the area of the sample-receiving zone is about 10 cm² or about 15 cm². In any embodiment, for a 1-5 milliliter volume of sample, the area of the sample-receiving zone is about 20 cm², about 25 cm², about 30 cm², about 31 cm², or about 25-35 cm².

The substrate 12 is water-proof, and is optionally a self-supporting water-proof substrate. In some embodiments, the substrate 12 is a film of a material such as polyester, polypropylene, silicone, or polystyrene, which will not absorb or otherwise be affected by water. Polyester films and polypropylene films having a thickness from about 20 micrometers to about 250 micrometers, as well as polystyrene films having a thickness of about 380 micrometers, have each been found to be suitable for the substrate 12. Other suitable substrates include paper with a polyethylene or other water-proof coating. An example of a suitable polyethylene-coated paper substrate is “Schoeller Type MIL” photoprint paper (commercially available from Schoeller Pulaski, New York). The substrate 12 may be either transparent or opaque, depending on whether one wishes to view bacterial colonies through the substrate. In some embodiments, the substrate 12 has a square grid pattern printed on the second major surface 12b to facilitate the counting of bacterial colonies.

The substantially dry, first or second microbial growth nutrient composition can include the microbial growth nutrient composition at a coating weight of 2 milligrams per square inch or more (mg/in²), 5 mg/in² or more, 10 mg/in² or more, 12 mg/in² or more, or 15 mg/in² or more; and at a coating weight of 50 mg/in² or less, 45 mg/in² or less, 40 mg/in² or less, 35 mg/in² or less, 30 mg/in² or less, 24 mg/in² or less, 22 mg/in² or less, 20 mg/in² or less, or 18 mg/in² or less. One suitable method for applying the microbial growth nutrient composition on the substrate includes preparing an aqueous solution or a suspension including at least the microbial growth nutrient composition, disposing a coating of the solution or suspension on the substrate surface, and drying the coating to form the substantially dry microbial growth nutrient composition. The skilled practitioner is capable of selecting a suitable coating method, including for instance and without limitation, knife-coating, gravure coating, curtain coating, air knife coating spray coating, die coating, draw bar coating or curtain coating or roll-coating. The coating is optionally dried at an elevated temperature (e.g., in a range from 50° C. to 100° C.) or in ambient conditions. In some embodiments, the microbial growth nutrient composition contains 75% by weight or more microbial growth nutrients, or 80% by weight or more, or 85% by weight or more, or 90% by weight or more, or 95% by weight or more microbial growth nutrients. Advantageously, in certain embodiments a greater amount of microbial growth nutrients can be included in the device than in devices in which the microbial growth nutrient composition is powder coated to an adhesive layer and/or combined with a substantial amount of a cold-water-soluble gelling agent.

The first or second adhesive composition can be (substantially) water-insoluble and non-inhibitory to the growth of microorganisms. In some embodiments, the first adhesive composition 16 is sufficiently transparent when wet to enable the viewing of bacterial colonies through the film coated with the adhesive. In some embodiments, the first or second adhesive composition can be a pressure-sensitive adhesive. In some other embodiments, heat-activated adhesives in which a lower melting substance is coated onto a higher melting substance may also be used. Water-activated adhesives such as mucilage may also be useful.

Suitable adhesives are transparent when wetted with water. As noted above, the adhesive composition is often water insoluble. In certain embodiments, the adhesive composition comprises a solvent based adhesive. The first adhesive composition and, if present, second adhesive composition often is a pressure sensitive adhesive. For instance, the adhesive may be a pressure-sensitive adhesive such as a water-insoluble adhesive comprising a copolymer of an alkyl acrylate monomer and an alkyl amide monomer or a copolymer of an alkyl acrylate monomer and an acrylic acid. Preferably the weight ratio of alkyl acrylate monomer to alkyl amide monomer in these copolymers is from about 90:10 to 99:1, more preferably 94:6 to 98:2. The alkyl acrylate monomer comprises a lower alkyl (C2 to C10) monomer of acrylic acid, including, for example, isooctyl acrylate (IOA), 2-ethylhexyl acrylate, butyl acrylate, ethyl acrylate, isoamyl acrylate, and mixtures thereof, while the alkyl amide monomer can comprise, without limitation, acrylamide (ACM), methacrylamide, N-vinylpyrrolidone (NVP), N-vinylcaprolactam (NVCL), N-vinyl-2-piperidine, N-(mono- or di-lower alkyl (C2 to C5))(meth)acrylamides, N-methyl(meth)acrylamide, N,N-dimethyl(meth) acrylamides, or mixtures thereof. Suitable adhesives may also include those described in U.S. Pat. Nos. 4,565,783, 5,089,413, 5,681,712, and 5,232,838. In some embodiments, silicone pressure sensitive adhesives may be used, including for example those described in U.S. Pat. Nos. 7,695,818 and 7,371,464.

In the present disclosure, the cover sheet 22 is usually selected to be transparent, in order to facilitate counting of microbial colonies, and is typically also selected to be impermeable to bacteria and have low moisture vapor transmission rate (i.e., the cover sheet 22 prevents undesired contamination of the dehydrated medium during shipping, storage and use of the devices and provides an environment which will support the growth of microorganisms during the incubation period). In some embodiments, the cover sheet 22 has the same properties (e.g., being water-proof) as the substrate 12. The cover sheet 22 can be selected to provide the amount of oxygen transmission necessary for the type of microorganism desired to be grown. For example, some polyester films have low oxygen permeability (less than 5 g/645 cm²/24 hours per 25 micrometers of thickness) and would be suitable for growing anaerobic bacteria. On the other hand, some polyethylenes have high oxygen permeability (e.g., approximately 500 g/645 cm²/24 hours per 25 micrometers of thickness) and would be suitable for aerobic organisms. Suitable material for the cover sheet 22 includes polypropylene, polyester, polyethylene, polystyrene, or silicone. In certain embodiments, the cover sheet 22 comprises oriented polypropylene, such as biaxially oriented polypropylene, which in some exemplary embodiments has a thickness of about 40 micrometers.

In certain embodiments, the cold-water-soluble hydrogel-forming composition contains one or more organic cold-water-soluble agents, such as alginate, carboxymethyl cellulose, tara gum, hydroxyethyl cellulose, hydroxypropyl methylcellulose, guar gum, locust bean gum, xanthan gum, polyacrylamide, polyurethane, polyethylene oxides. Combinations of natural and/or synthetic gelling agents are contemplated. Preferred gelling agents include guar gum, xanthan gum, and locust bean gum, these gelling agents being useful individually or, in any embodiment, in combination with one another. A uniform monolayer of a cold-water-soluble hydrogel-forming composition is desired with sufficient surface area exposed for hydration. In any embodiment, the first and/or second cold-water-soluble hydrogel-forming composition comprises a mixture of gelling agents. Optionally, the powdered cold-water-soluble hydrogel-forming composition may further comprise an inducer, and indicator agent, or a combination of these.

Fluid control film can include those described in US 2017/0045284 A1 (Meuler et al.).

For example, the fluid control film includes fluid control channels extending along a channel longitudinal axis. Each of the fluid control channels has a surface and is configured to allow capillary movement of liquid in the channels. In some embodiments, the fluid control film can further include a hydrophilic surface treatment covalently bonded to at least a portion of the surface of the fluid control channels. In some other embodiments, the fluid control film can have a noncovalent hydrophilic surface treatment, for example, surfactant treatment, disposed to a least a portion of the surface of the fluid control channels. The fluid control film exhibits a capillary rise percent recovery of at least 10%. Typically, the hydrophilic surface treatment includes functional groups selected from a non-zwitterionic sulfonate, a non-zwitterionic carboxylate, a zwitterionic sulfonate, a zwitterionic carboxylate, a zwitterionic phosphate, a zwitterionic phosphonic acid, a zwitterionic phosphonate, or a combination thereof.

The fluid control films according to the present disclosure comprise a microstructured surface having a plurality of microreplicated structures. Fluid control films may have a variety of topographies. Exemplary fluid control films are comprised of a plurality of channels with V-shaped or rectangular cross-sections, and combinations of these, as well as structures that have channels, secondary channels, i.e., channels within channels. Additionally, the topography may include microstructured posts and protrusions.

The channels on the microstructured surface have channel ends. In certain embodiments, the fluid control film may include a removing means. The removing means generally withdraws fluid from the channels adjacent one of the channel ends. In another embodiment, the removing means withdraws the fluid from the channels adjacent both channel ends. The removing means may include an absorbent material disposed in communication with the channels. In one embodiment, the removing means includes a fluid drip collector.

Generally, the channels in the microstructure are defined by generally parallel ridges including a first set of ridges having a first height and a second set of ridges having a second, taller height. An upper portion of each ridge of the second set of ridges may have a lower melting temperature than a lower portion thereof. The channels have a pattern geometry selected from the group consisting of linear, curvilinear, radial, parallel, nonparallel, random, or intersecting.

In some embodiments, the fluid control film has a contact angle less than 90 degree. The contact angle Theta (θ) is the angle between a line tangent to the surface of a bead of fluid on a surface at its point of contact to the surface and the plane of the surface. A bead of fluid whose tangent was perpendicular to the plane of the surface would have a contact angle of 90 degrees. Typically, if the contact angle is 45 degrees or less, the solid surface is considered to be wet by the fluid. Surfaces on which drops of water or aqueous solutions exhibit a contact angle of less than 45 degrees are commonly referred to as “hydrophilic”. As used herein, “hydrophilic” is used only to refer to the surface characteristics of a material, i.e., that it is wet by aqueous solutions, and does not express whether or not the material absorbs aqueous solutions. Accordingly, a material may be referred to as hydrophilic whether or not a sheet of the material is impermeable or permeable to aqueous solutions. Thus, hydrophilic films used in the present application may be formed from films prepared from resin materials that are inherently hydrophilic, such as for example, poly(vinyl alcohol). Fluids which yield a contact angle of near zero on a surface are considered to completely wet out the surface. Polyolefins, however, are typically inherently hydrophobic, and the contact angle of a polyolefin film, such as polyethylene or polypropylene, with water is typically greater than 90 degrees.

Generally, the channels in the microstructure are defined by generally parallel ridges including a first set of ridges having a first height and a second set of ridges having a second, taller height. An upper portion of each ridge of the second set of ridges may have a lower melting temperature than a lower portion thereof. The channels have a pattern geometry selected from the group consisting of linear, curvilinear, radial, parallel, nonparallel, random, or intersecting.

FIG. 2 is a cross section of a fluid control film 200 according to an exemplary embodiment. The fluid control film 200 comprises a fluid control film layer 201 having primary and secondary channels 230, 231 defined by primary and secondary ridges 220, 221, wherein the channels 230, 231 and ridges 220, 221 run along a channel axis that makes an angle, θ, with respect to the longitudinal axis of the fluid control film layer 201, e.g., the x-axis. Each primary channel 230 is defined by a set of primary ridges 220 (first and second) on either side of the primary channel 230. The primary ridges 220 have a height hₚ that is measured from the bottom surface 230a of the channel 230 to the top surface 220a of the ridges 220. In some embodiments, microstructures are disposed within the primary channels 230. In some embodiments, the microstructures comprise secondary channels 231 disposed between the first and secondary primary ridges 220 of the primary channels 230. Each of the secondary channels 231 is associated with at least one secondary ridge 221. The secondary channels 231 may be located between a set of secondary ridges 221 or between a secondary ridge 221 and a primary ridge 220.

The center-to-center distance between the primary ridges, dₚᵣ, may be in a range of about 25 micrometers to about 3000 micrometers; the center-to-center distance between a primary ridge and the closest secondary ridge, dₚₛ may be in a range of about 5 micrometers to about 350 micrometers; the center-to-center distance between two secondary ridges, dₛₛ, may be in a range of about 5 micrometers to about 350 micrometers. In some cases, the primary and/or secondary ridges may taper with distance from the base.

The distance between external surfaces of a primary ridge at the base, d_{pb}, may be in a range of about 15 micrometers to about 250 micrometers and may taper to a smaller distance of dₚₜ in a range of about 1 micrometers to about 25 micrometers. The distance between external surfaces of a secondary ridge at the base, d_{sb}, may be in a range of about 15 micrometers to about 250 micrometers and may taper to a smaller distance of dₛₜ in a range of about 1 micrometers to about 25 micrometers. In one example, dₚᵣ=0.00898 inches (228 micrometers), dₚₛ=0.00264 inches (67 micrometers), dₛₛ=0.00185 inches (47 micrometers), d_{pb}=0.00251 inches (64 micrometers), dₚₜ=0.00100 inches (25 micrometers), d_{sb}=0.00131 inches (33 micrometers), dₛₜ=0.00100 inches (25 micrometers), hₚ=0.00784 inches (199 micrometers), and hₛ=0.00160 inches (41 micrometers).

The secondary ridges have height hₛ that is measured from the bottom surface 230a of the channel 230 to the top surface 221a of the secondary ridges 221. The height hₚ of the primary ridges 220 is often greater than the height hₛ of the secondary ridges 221. In some embodiments the height of the primary ridges is between about 25 micrometers to about 3000 micrometers and the height of the secondary ridges is between about 5 micrometers to about 350 micrometers. In some embodiments, a ratio of the secondary ridge 221 height hₛ to the primary ridge 220 height hₚ is about 1:5. The primary ridges 220 can be designed to provide durability to the fluid control film layer 200 as well as protection to the secondary channels 231, secondary ridges and/or or other microstructures disposed between the primary ridges 220.

The fluid control film 200 optionally has an adhesive layer 205 disposed on the bottom surface 201a of the fluid control film layer 201. The adhesive layer 205 may allow the fluid control film layer 200 to be attached to some external surface 202 to help manage liquid dispersion across the external surface. The combination of an adhesive layer 205 and the fluid control film layer 201 forms a fluid control tape. The adhesive layer 205 may be continuous or discontinuous.

The fluid control film layer 201 is configured to disperse fluid across the surface of the fluid control film layer 201 to facilitate evaporation of the fluid. In some embodiments, the adhesive layer 205 may be or comprise a hydrophobic material that repels liquid at the interface 202a between the adhesive layer 205 and the external surface 202, reducing the collection of liquid at the interface 202a.

The adhesive layer 205 has a thickness tₐ and the fluid control film layer 201 has a thickness tᵥ from the bottom surface 230a of the channels 230, 231 to the bottom surface 201a of the fluid control film layer 201. In some embodiments, the total thickness between the bottom surface 230a of the channels 230, 231 and the bottom surface 205a of the adhesive layer 205, tᵥ+tₐ can be less than about 300 micrometers, e.g., about 225 micrometers. This total thickness tᵥ+tₐ may be selected to be small enough to allow liquid to be efficiently wicked from the external surface 202 through the channel openings at the edges of the fluid control film layer 201 and into the channels 230, 231.

A method of detecting and enumerating at least one microorganism in a sample is provided. The method includes providing a device according to the current disclosure, adding a predetermined volume of a sample containing at least one microorganism into the aperture 20 of the spacer element 19 to form an inoculated device, contacting the cover sheet to the substrate, incubating the inoculated device, and detecting the presence or an absence of a colony of the target microorganism in the device. The cold-water-soluble hydrogel-forming composition on the cover sheet is hydrated and forms a hydrogel when an aqueous sample is placed into the device and the hydrogel can self-spread and fills the channels of the flow control film. It has been unexpectedly discovered that the colonies would form such punctate colonies and not grow along a channel longitudinal axis of channels of the fluid control film.

The method further comprises a step of incubating the device for a period of time at a temperature that facilitates growth and detection of a target microorganism. A person having ordinary skill in the art will recognize the incubation temperature and period of time will depend upon a number of factors (e.g., the target microorganism, nutrients present in the sample, nutrients present in the device, inhibitory agents present in the sample and/or the device) and will adjust the incubation time and temperature accordingly.

The method further comprises a step of detecting a presence or an absence of a colony of the target microorganism in the device. In any embodiment, detecting a presence or an absence of a colony of the target microorganism in the device can comprise detecting a colony (e.g., visually or using machine vision) in the first compartment of the device. In any embodiment, detecting a presence or an absence of a colony of the target microorganism in the device can comprise detecting a change associated with the indicator reagent. The indicator reagent may change from a first state (e.g., substantially colorless or nonfluorescent) to a second state (e.g., colored or fluorescent) in and/or surrounding a colony of the target microorganism. In any embodiment, the colonies can be enumerated and, optionally, the number of colonies of target microorganisms can be recorded. In some embodiments, the microorganisms can be counted using an automated system, such as an automated colony counter.

The following embodiments are intended to be illustrative of the present disclosure and not limiting.

### Embodiments

Embodiment 1 is a device for growing microorganisms, comprising: a body member comprising a self-supporting, water-proof substrate having upper and lower surfaces; a hydrophobic spacer element adhered to the upper surface of the substrate forming side walls to retain a predetermined amount of liquid in contact with the substrate, wherein the hydrophobic spacer element has a hole therein; a fluid control film in the hole of the hydrophobic spacer element;

a cover sheet having an inner-facing surface and an outer-facing surface, the cover sheet adhered to at least a portion of the body member; and a substantially dry, first microbial growth nutrient composition disposed on a portion of the inner surface of the cover sheet; a first adhesive composition adhered to the first microbial growth nutrient composition; and a cold-water-soluble first hydrogel-forming composition adhered to the first adhesive composition.

Embodiment 2 is the device of embodiment 1, wherein the fluid control film comprises a plurality of microreplicated structures.

Embodiment 3 is the device of any of embodiments 1 to 2, wherein the fluid control film comprises a plurality of fluid control channels extending along a channel longitudinal axis, each of the fluid control channels comprising a surface and configured to allow capillary movement of liquid in the channels.

Embodiment 4 is the device of any of embodiments 1 to 3, wherein the fluid control film comprises a hydrophilic surface treatment covalently bonded to at least a portion of the surface of the fluid control channels.

Embodiment 5 is the device of any of embodiments 1 to 4, wherein the fluid control film comprise a noncovalent hydrophilic surface treatment disposed to a least a portion of the surface of the fluid control channels.

Embodiment 6 is the device of any of embodiments 1 to 5, wherein the fluid control film has a contact angle less than 90 degree.

Embodiment 7 is the device of any of embodiments 1 to 6, further comprising a second adhesive composition adhered to the upper surface of the self-supporting waterproof substrate, wherein the second adhesive composition is in between the hydrophobic spacer element and the substrate.

Embodiment 8 is the device of any of embodiments 1 to 7, wherein the spacer element comprises a hydrophobic foam sheet.

Embodiment 9 is the device of embodiment 8, wherein the hydrophobic foam is polystyrene or polyethylene foam.

Embodiment 10 is the device of any of embodiments 1 to 9, wherein the cover sheet comprises a transparent film.

Embodiment 11 is the device of embodiment 10, wherein the film is selected from the group consisting of polyester, polyethylene, polypropylene, polystyrene and silicone.

Embodiment 12 is the device of any of embodiments 1 to 11, wherein the substrate is a film selected from the group consisting of polyester, polypropylene, polyethylene and polystyrene.

Embodiment 13 is the device of any of embodiments 1 to 12, wherein the gelling agent is selected from the group consisting of xanthum gum, guar gum, locust bean gum, carboxymethyl cellulose, hydroxyethyl cellulose, and algin.

Embodiment 14 is a method comprising: providing a device according to any of embodiments 1 to 13; adding a predetermined volume of a sample containing at least one microorganism into the device to form an inoculated device; contacting the cover sheet to the self-supporting, water-proof substrate; incubating the inoculated device; and detecting the presence or an absence of a colony of the target microorganism in the device.

The following working examples are intended to be illustrative of the present disclosure and not limiting.

### EXAMPLES

TABLE 1
Materials

| Material Name/Description | Source |
|---|---|
| BACTO Tryptic Soy Broth (TSB) | Becton, Dickinson and |
| | Company, Franklin Lakes, NJ |
| Guar Gum (Meyprogat 150) | Danisco, Copenhagen, Denmark |
| 2,3,5-Triphenyl Tetrazolium | Sigma-Aldrich Corporation, |
| Chloride (TTC) | St. Louis, MO |
| TRITON X-100 (4-(1,1,3,3- | Sigma-Aldrich Corporation, |
| Tetramethylbutyl)phenyl-polyethylene | St. Louis, MO |
| glycol); CAS No. 9002-93-1 | |

### Incubation and Inoculation

The bacterial strain Escherichia coli (ATCC 25922) was obtained from Microbiologics Incorporated (St. Cloud, Minn.) and incubated overnight in tryptic soy broth (TSB) at 37° C. and 200 rpm in an INNOVA44 incubator (New Brunswick Scientific, Enfield, Conn.). The inoculum was prepared by serially diluting the culture sample with Butterfield's Buffer (3M Corporation, St. Paul, Minn.). The culture sample was diluted so as to yield a final concentration of about 50-250 colony forming unit (cfu) counts per 1 mL of inoculum.

### Preparative Example 1. Fluid Control Film Fabrication

Fluid control film of FIG. 2 was prepared according to the extrusion embossing procedure described in US Patent Application 20017/0045284 (Meuler), incorporated by reference in its entirety. Using the designators from FIG. 2, the fluid control film of the examples had the following dimensions: dpr=0.00898 inches (228 microns), dps=0.00264 inches (67 microns), dss=0.00185 inches (47 microns), dpb=0.00251 inches (64 microns), dpt=0.00100 inches (25 microns), dsb=0.00131 inches (33 microns), dst=0.00100 inches (25 microns), hp=0.00784 inches (199 microns), hs=0.00160 inches (41 microns). The film was made from a low density polyethylene polymer (obtained under the trade designation “DOW LDPE 9551” from the Dow Chemical Company, Midland, Mich.).

### Preparative Example 2. Plasma Treatment of Fluid Control Films

A silicon containing film layer [methods of forming described in U.S. Pat. No. 6,696,157 (David) and 8664323 (Iyer) and US Patent Application 2013/0229378 (Iyer)] was applied to the fluid control film of Preparative Example 1 using a Plasma-Therm 3032 batch plasma reactor (obtained from Plasma-Therm LLC, St. Petersburg, Fla.). The instrument was configured for reactive ion etching with a 26 inch lower powered electrode and central gas pumping. The chamber was pumped with a roots type blower (model EH1200 obtained from Edwards Engineering, Burgess Hill, UK) backed by a dry mechanical pump (model iQDP80 obtained from Edwards Engineering). The RF power was delivered by a 3 kW, 13.56 Mhz solid-state generator (RFPP model RF30S obtained from Advanced Energy Industries, Fort Collins, Colo.). The system had a nominal base pressure of 5 mTorr. The flow rates of the gases were controlled by MKS flow controllers (obtained from MKS Instruments, Andover, Mass.).

Samples of fluid control film were fixed on the powered electrode of the plasma reactor. After pumping down to the base pressure, the gases tetramethylsilane (TMS) and oxygen (O₂) were introduced at varying flow rates (see Table 2). Once the gas flows stabilized in the reactor, rf power (1000 watts) was applied to the electrode to generate the plasma. The plasma exposure time was also varied (see Table 2). Following completion of the plasma treatment, the chamber was vented to the atmosphere and the treated fluid control film was removed from the chamber.

TABLE 2
Plasma Treatment Conditions to Prepare Fluid Control Films A-D

| | TMS | Oxygen | Plasma |
|---|---|---|---|
| | Flow | Flow | Deposition |
| Fluid Control Film | Rate | Rate | Time |
| Designation | (sccm) | (sccm) | (seconds) |
| Fluid Control Film A | 75 | 750 | 60 |
| Fluid Control Film B | 75 | 1100 | 60 |
| Fluid Control Film C | 75 | 925 | 60 |

| | |
|---|---|
| Fluid Control Film D | Film D was plasma treated in a two step process |
| | as follows: Step 1. TMS flow rate at 150 sccm, |
| | oxygen flow rate at 500 sccm, and deposition |
| | time of 30 seconds; Step 2. oxygen at a |
| | flow rate of 500 sccm for 20 seconds |

### Preparative Example 3. Fluid Control Film Containing a Surfactant

A fluid control film was prepared according to the description of Preparative Example 1 with the exception that 0.5 weight % of the nonionic surfactant TRITON-X100 was incorporated in the low density polyethylene polymer used in the extrusion embossing process. The resulting fluid control film was designated as Fluid Control Film E.

### Example 1. Microbial Detection Devices

Microbial detection devices according to the device of FIG. 1 were constructed. For each device, the substrate of the body member was a clear, biaxially-oriented polypropylene (BOPP) film (1.6 mil (0.04 mm) thick and corona treated on both sides) that was cut into 76 mm wide by 102 mm long sections. The body member was completed by adhesively laminating a 76 mm wide by 102 mm long polyethylene film spacer (Optimum Plastics, Bloomer, Wis.) to one side of the substrate. The spacer was approximately 20 mil (0.51 mm) thick and contained a circular hole (5.1 cm diameter) that was positioned near the center of the spacer. The circular hole defined the perimeter of the sample-receiving zone of the device. A circular section of fluid control film (selected from fluid control films designated A-E in Table 2) was cut and sized to fit in the hole (5.1 cm diameter) and oriented so that the non-microreplicated surface of the film was adhesively laminated to the exposed substrate surface defined by the hole.

The cover sheet of the device was a clear, biaxially-oriented polypropylene (BOPP) film (1.6 mil (0.04 mm) thick and corona treated on both sides) that was sequentially coated on one side with a microbial growth nutrient composition, an adhesive composition, and a guar gum (e.g. cold-water-soluble hydrogel-forming) composition according to the following procedure.

The microbial growth nutrient coating composition was prepared by vigorously mixing (using an air-driven overhead mixer with a JIFFY-type mixing impeller) 30 g of tryptic soy broth (TSB) and 500 mL of purified water [obtained from a MILLI-Q Gradient Water Purification System (model #ZMQS6V00Y,

Merck Millipore Corporation, Billerica, Mass.)] until the TSB was completely dissolved. The resulting solution had a pH of 7.3 (Mettler-Toledo FE20 FIVEEASY pH Meter, Mettler-Toledo LLC, Columbus, Ohio). Guar gum (10 g) was added to the nutrient solution and vigorous stirring was continued for about 10 minutes. The resulting solution was knife-coated onto one side of the BOPP cover sheet film with a 14 mil (0.35 mm) gap setting. The nutrient coated film was dried in an oven at 85° C. for 12 minutes to provide a dry coat weight of about 360 mg/24 in² (2.3 mg/cm²).

An isooctyl acrylate/acrylic acid (98/2 weight ratio) pressure-sensitive adhesive (PSA) coating formulation containing TTC (2,3,5-triphenyl tetrazolium chloride) indicator as described in Example 4 of U.S. Pat. No. 5,409,838 (which is incorporated herein by reference) was knife-coated onto the exposed nutrient coating with a 2 mil (0.05 mm) gap setting. The resulting coated film was dried in an oven at 65° C. for 6 minutes to provide a PSA coating having a dry coat weight of about 180 mg/24 in² (1.15 mg/cm²). The adhesive coated side of the cover sheet film was then powder coated with guar gum. The powder was evenly applied and excess powder was removed from the adhesive layer by hand shaking of the film followed by lightly brushing the surface with a paper towel. The final coat weight of the guar gum was about 400 mg/24 in² (2.6 mg/cm²).

The coated cover sheet film was then cut to match the dimensions of the body member. The finished devices were assembled by attaching a cover sheet to a body member (in a hinge-like fashion) along one edge (the 76 mm edge) of the spacer using double sided adhesive tape. For each device, the cover sheet and the body member were oriented so that the coated surface of the cover sheet faced the spacer side of the body member.

The finished detection device was inoculated with the E. coli inoculum. The cover sheet of the device was lifted and 1 mL of the inoculum (i.e., final dilution as described above) was added by pipet across the fluid control film so that the channels were filled with liquid. The cover sheet was gently returned to its original position. All devices demonstrated self-spreading of the water in the channels so that the guar gum was evenly wetted and formed a hydrogel that filled the channels of the fluid control film. The devices were incubated at 37° C. for 24 hours. At the end of the incubation period, the red-colored colonies were counted by visual examination. For all of the devices, punctate colonies were observed disposed across the surface of the hydrogel. The results are presented in Table 3.

TABLE 3

| | Colony (cfu) | Self-Spreading | Punctate |
|---|---|---|---|
| Device Containing | Count | of Hydrogel | Colonies |
| Fluid Control Film A | 202 | yes | yes |
| Fluid Control Film B | 144 | yes | yes |
| Fluid Control Film C | 214 | yes | yes |
| Fluid Control Film D | 154 | yes | yes |
| Fluid Control Film E | 180 | yes | yes |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A device for growing microorganisms, comprising:
a body member comprising a self-supporting, water-proof substrate having upper and lower surfaces;
a fluid control film on the upper surface of the self-supporting, water-proof substrate;
a cover sheet having an inner-facing surface and an outer-facing surface, the cover sheet adhered to at least a portion of the body member; and
a substantially dry, first microbial growth nutrient composition disposed on a portion of the inner surface of the cover sheet;
a first adhesive composition adhered to the first microbial growth nutrient composition; and
a cold-water-soluble first hydrogel-forming composition adhered to the first adhesive composition.

2. The device of claim 1, wherein the fluid control film comprises a plurality of microreplicated structures.

3. The device of claim 1, wherein the fluid control film comprises a plurality of fluid control channels extending along a channel longitudinal axis, each of the fluid control channels comprising a surface and configured to allow capillary movement of liquid in the channels.

4. The device of claim 1, wherein the fluid control film comprises a hydrophilic surface treatment covalently bonded to at least a portion of the surface of the fluid control channels.

5. The device of claim 1, wherein the fluid control film comprise a noncovalent hydrophilic surface treatment disposed to a least a portion of the surface of the fluid control channels.

6. The device of claim 1, wherein the fluid control film has a contact angle less than 90 degree.

7. The device of claim 1, further comprising a second adhesive composition adhered to the upper surface of the self-supporting waterproof substrate, wherein the second adhesive composition is in between the hydrophobic spacer element and the substrate.

8. The device of claim 1, wherein the spacer element comprises a hydrophobic foam sheet.

9. The device of claim 8, wherein the hydrophobic foam is polystyrene or polyethylene foam.

10. The device of claim 1, wherein the cover sheet comprises a transparent film.

11. The device of claim 10, wherein the film is selected from the group consisting of polyester, polyethylene, polypropylene, polystyrene and silicone.

12. The device of claim 1, wherein the substrate is a film selected from the group consisting of polyester, polypropylene, polyethylene and polystyrene.

13. The device of claim 1, wherein the gelling agent is selected from the group consisting of xanthum gum, guar gum, locust bean gum, carboxymethyl cellulose, hydroxyethyl cellulose, and algin.

14. The device of claim 1, further comprising a hydrophobic spacer element adhered to the upper surface of the substrate forming side walls to retain a predetermined amount of liquid in contact with the substrate, wherein the hydrophobic spacer element has a hole therein and wherein the fluid control film is in the hole of the hydrophobic spacer element.

15. A method comprising:
providing a device according to claim 1;
adding a predetermined volume of a sample containing at least one microorganism into the device to form an inoculated device;
contacting the cover sheet to the self-supporting, water-proof substrate;
incubating the inoculated device; and
detecting the presence or an absence of a colony of the target microorganism in the device.
